(19) United States
(12) Patent Application Publication
(10) Pub. No.: US 2010/0297241 A1
(43) Pub. Date: Nov. 25, 2010

(54) Amorphous Fesoterodine Fumarate

(75) Inventors: Kishore Charugundla, Andhra Pradesh (IN); Udhaya Kumar Chandramohan, Pondicherry (IN); Nitin Sharadchandra Pradhan, Maharashtra (IN); Jon Valgeirsson, Hafnarfjordur (IS); Praveen Kumar Neela, Andhra Pradesh (IN)

Correspondence Address: CANTOR COLBURN LLP, 20 Church Street, 22nd Floor, Hartford, CT, 06103, US

(73) Assignee: ACTAVIS GROUP PTC EHF, Hafnarfjordur (IS)

(21) Appl. No.: 12/680,848

(22) PCT Filed: Oct. 1, 2008
(86) PCT No.: PCT/IB2008/003105

§ 371 Date: Aug. 10, 2010

(30) Foreign Application Priority Data
Oct. 1, 2007 (IN) 2206/CHE/2007

(51) Int. Cl. A61K 9/14; C07C 69/66; A61K 31/222; A61P 13/02

(52) U.S. Cl. 424/489; 560/250; 514/546

(57) ABSTRACT

The present invention provides a novel amorphous form of fesoterodine fumarate, process for preparation, pharmaceutical compositions, and method of treating thereof.

### CROSS REFERENCE TO RELATED APPLICATION

This application claims the benefit of priority to Indian provisional application No. 2206/CHE/2007, filed on Oct. 1, 2007, which is incorporated herein by reference.

### FIELD OF THE INVENTION

The present invention provides a novel amorphous form of fesoterodine fumarate, process for preparation, pharmaceutical compositions, and method of treating thereof.

### BACKGROUND OF THE INVENTION

U.S. Pat. No. 6,713,464 B1 disclosed a variety of 3,3-diphenylpropylamine derivatives, processes for their preparation, pharmaceutical compositions in which they are present and method of use thereof. These compounds are anti-muscarinic agents with superior pharmacokinetic properties compared to existing drugs such as oxybutynin and tolterodine and useful in the treatment of urinary incontinence, gastrointestinal hyperactivity (irritable bowel syndrome) and other smooth muscle contractile conditions. Among them, Fesoterodine fumarate, chemically 2-[(1R)-3-[bis(1-methylethyl)amino]-1-phenylpropyl]-4-hydroxymethylphenylisobutyrate ester hydrogen fumarate is a new, potent and competitive muscarinic antagonist and useful in the potential treatment of urinary incontinence. Fesoterodine fumarate is represented by the following structural formula I:

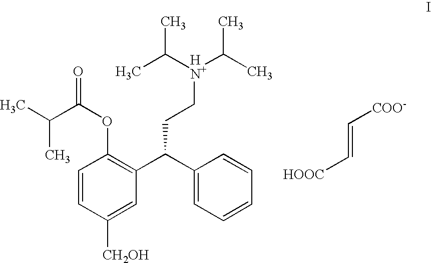

Processes for the preparation of fesoterodine and related compounds, and their pharmaceutically acceptable salts were disclosed in the U.S. Pat. Nos. 6,713,464 B1 and 6,858,650 B1; U.S. Patent Application No. 2006/0270738 and PCT Publication No. WO 2007/138440 A1.

According to the U.S. Pat. No. 6,713,464 B1 (herein after referred to as the '464 patent), fesoterodine was prepared by the reaction of (+)-6-bromo-4-phenylchroman-2-one with benzyl chloride in the presence of sodium iodide and anhydrous potassium carbonate in methanol and acetone to give (+)-3-(2-benzyloxy-5-bromophenyl)-3-phenylpropionic acid methyl ester as a light yellow oil, which by reduction with lithium aluminum hydride in tetrahydrofuran at room temperature to give (+)-3-(2-benzyloxy-5-bromophenyl)-3-phenylpropan-1-ol, which is then treated with p-toluenesulphonyl chloride in the presence of pyridine in dichloromethane to afford (+)-toluene-4-sulphonic acid 3-(2-benzyloxy-5-bromophenyl)-3-phenylpropyl ester followed by reaction with N,N-diisopropylamine in acetonitrile at reflux temperature to give (+)-[3-(2-benzyloxy-5-bromophenyl)-3-phenylpropyl]-diisopropylamine as a brown and viscous syrup, followed by resolution with D-(−)-tartaric acid to afford (R)[3-(2-benzyloxy-5-bromophenyl)-3-phenylpropyl]-diisopropylamine, which is then subjected to Grignard reaction with ethylbromide and magnesium in the presence of solid carbon dioxide in tetrahydrofuran to produce (R)-4-benzyloxy-3-(3-diisopropylamino-1-phenylpropyl)-benzoic acid hydrochloride followed by esterification with methanol in the presence of sulphuric acid to produce (R)-4-benzyloxy-3-(3-diisopropylamino-1-phenylpropyl)-benzoic acid methyl ester, which is then reduced with lithium aluminum hydride to give (R)[4-benzyloxy-3-(3-diisopropylamino-1-phenylpropyl)-phenyl]-methanol, which is then subjected to deprotection with Raney-Nickel to give (R)-2-(3-diisopropylamino-1-phenylpropyl)-4-hydroxymethylphenol followed by condensation with isobutyryl chloride in an inert solvent in the presence of a base to give fesoterodine.

The '464 patent mentions that some of the disclosed compounds which can form a salt with physiologically acceptable organic and inorganic acids like hydrochloride, hydrobromide, only the hydrochloride salts of the disclosed compounds have been prepared.

U.S. Pat. No. 6,858,650 B1 (herein after referred to as the '650 patent) discloses crystalline compounds of 3,3-diphenylpropylamine derivatives in the form of their salts, including, among other things, crystalline fesoterodine fumarate.

Polymorphism is defined as “the ability of a substance to exist as two or more crystalline phases that have different arrangement and/or conformations of the molecule in the crystal lattice. Thus, in the strict sense, polymorphs are different crystalline forms of the same pure substance in which the molecules have different arrangements and/or configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and infrared spectrometry (IR).

Solvent medium and mode of isolation play very important role in obtaining a polymorphic form over the other.

It has been disclosed in the art that the amorphous forms in a number of drugs exhibit different dissolution characteristics and in some cases different bioavailability patterns compared to crystalline forms [Konne T., Chem. Pharm. Bull., 38, 2003-2007 (1990)]. For some therapeutic indications one bioavailability pattern may be favored over another.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

Accordingly, there remains a need in the art for a novel, stable and substantially pure amorphous form of fesoterodine fumarate.

### SUMMARY OF THE INVENTION

The present inventors have now surprisingly and unexpectedly discovered a novel amorphous form of fesoterodine fumarate with high purity, adequate stability and good dissolution properties.

In one aspect, the present invention provides a novel and stable amorphous form of fesoterodine fumarate and use thereof for the preparation of fesoterodine or a pharmaceutically acceptable salt thereof.

In another aspect, the present invention further encompasses a process for preparing the highly pure and stable amorphous form of fesoterodine fumarate.

In another aspect, the present invention provides a pharmaceutical composition comprising amorphous fesoterodine fumarate of the present invention and one or more pharmaceutically acceptable excipients.

In still another aspect, the present invention provides a pharmaceutical composition comprising amorphous fesoterodine fumarate made by the process of the present invention, and one or more pharmaceutically acceptable excipients.

In still further aspect, the present invention further encompasses a process for preparing a pharmaceutical formulation comprising combining amorphous fesoterodine fumarate with one or more pharmaceutically acceptable excipients.

In another aspect, the amorphous fesoterodine fumarate disclosed herein for use in the pharmaceutical compositions of the present invention, wherein 90 volume-percent of the particles (D₉₀) have a size of less than or equal to about 500 microns, specifically less than or equal to about 300 microns, more specifically less than or equal to about 200 microns, still more specifically less than or equal to about 100 microns, and most specifically less than or equal to about 15 microns.

Unless otherwise indicated, the following definitions are set forth to illustrate and define the meaning and scope of the various terms used to describe the invention herein.

The term “polymorphic form” refers to a crystal modification that can be characterized by analytical methods such as X-ray powder diffraction, IR-spectroscopy, differential scanning calorimetry (DSC) or by its melting point.

The term “amorphous” means a solid without long-range crystalline order. Amorphous form of fesoterodine fumarate in accordance with the present invention preferably contains less than about 10% crystalline forms of fesoterodine fumarate, more preferably less than 5% crystalline forms of fesoterodine fumarate, and still more preferably is essentially free of crystalline forms of fesoterodine fumarate. “Essentially free of crystalline forms of fesoterodine fumarate” means that no crystalline polymorph forms of fesoterodine fumarate can be detected within the limits of a powder X-ray diffractometer.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The expression “pharmaceutically acceptable salt ” is meant those salts which are, within the scope of sound medical judgment, suitable for use in contact with the tissues of humans and lower animals without undue toxicity, irritation, allergic response and the like, commensurate with a reasonable benefit/risk ratio, and effective for their intended use.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia alginic acid, tragacanth, carboxymethylcellulose sodium, polyvinylpyrrolidone, compressible sugar (e.g., NuTab), ethylcellulose, gelatin, liquid glucose, methylcellulose, povidone and pregelatinized starch, combinations thereof and other material known to those of ordinary skill in the art. If required, other binders may also be included in the present invention.

Exemplary binders include starch, polyethylene glycol, guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, polypropylene glycol, polyoxyethylene-polypropylene copolymer, polyethylene ester, polyethylene sorbitan ester, polyethylene oxide, microcrystalline cellulose, polyvinylpyrrolidone, combinations thereof and other such materials known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, macrocrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

As used herein, D_{X} means that X percent of the particles have a diameter less than a specified diameter D. Thus, a D₉₀ or d(0.9) of less than 300 microns means that 90 volume-percent of the micronized particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

By “substantially pure” is meant having purity greater than about 99%, specifically greater than about 99.5%, and more specifically greater than about 99.9% measured by HPLC.

### BRIEF DESCRIPTION OF THE DRAWING

FIG. 1 is a characteristic powder X-ray diffraction (XRD) pattern of amorphous fesoterodine fumarate.

FIG. 2 is a characteristic infra red (IR) spectrum of amorphous fesoterodine fumarate

The X-Ray powder diffraction was measured by an X-ray powder diffractometer equipped with a Cu-anode (?=1.54 Angstrom), X-ray source operated at 40 kV, 40 mA and a Ni filter is used to strip K-beta radiation. Two-theta calibration is performed using an NIST SRM 1976, Corundum standard. The sample was analyzed using the following instrument parameters: measuring range=3-45° 2?; step width=0.01579° ; and measuring time per step=0.11 second.

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 3800 to 650 cm⁻¹.

### DETAILED DESCRIPTION OF THE INVENTION

According to one aspect of the present invention, there is provided a stable and substantially pure amorphous form of fesoterodine fumarate.

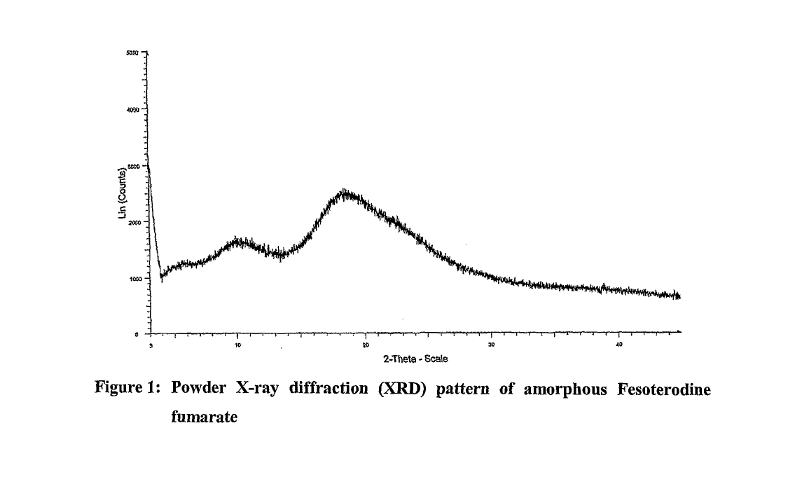

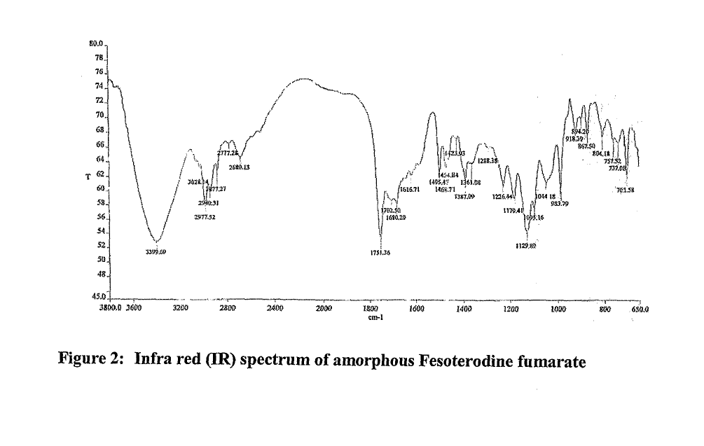

Amorphous form of fesoterodine fumarate is characterized by at least one, and preferably all, of the following properties: a powder XRD pattern substantially in accordance with FIG. 1; an IR spectrum substantially in accordance with FIG. 2; and an IR spectrum having absorption bands at about 3399, 3028, 2977, 2940, 2877, 1751, 1702, 1680, 1616, 1495, 1468, 1387, 1226, 1179, 1129, 1095, 983, 867, 804 and 701±1 cm⁻¹ substantially as depicted in FIG. 2. The X-ray powder diffraction pattern shows no peaks, thus demonstrating the amorphous nature of the product.

According to another aspect of the present invention, a process is provided for preparation of a stable and substantially pure amorphous form of fesoterodine fumarate, which comprises:

a) providing a solution of fesoterodine fumarate in a suitable solvent or a mixture of solvents capable of dissolving fesoterodine fumarate;
b) optionally, filtering the solvent solution to remove any extraneous matter; and
c) substantially removing the solvent from the solution to afford amorphous form of fesoterodine fumarate.

The process can produce amorphous fesoterodine fumarate in substantially pure form.

The term “substantially pure amorphous form of fesoterodine fumarate” refers to the amorphous form of fesoterodine fumarate having purity greater than about 98%, specifically greater than about 99%, more specifically greater than about 99.5% and still more specifically greater than about 99.9% (measured by HPLC).

The amorphous fesoterodine fumarate obtained by the process disclosed herein is stable, consistently reproducible and has good flow properties, and which is particularly suitable for bulk preparation and handling, and so, the amorphous fesoterodine fumarate obtained by the process disclosed herein is suitable for formulating fesoterodine fumarate.

The suitable solvent used in step-(a) is selected from the group comprising water, alcohols, ketones, chlorinated hydrocarbons, hydrocarbons, nitriles, esters, cyclic ethers, aliphatic ethers, polar aprotic solvents, and mixtures thereof. Preferable solvents are chlorinated hydrocarbons, ketones, alcohols and mixtures thereof, more preferably a mixture of methylene chloride with an alcohol solvent, and most preferably a mixture of methylene chloride and methanol.

Exemplary alcohol solvents include, but are not limited to, C₁ to C₈ straight or branched chain alcohol solvents such as methanol, ethanol, propanol, butanol, amyl alcohol, hexanol, and mixtures thereof. Specific alcohol solvents are methanol, ethanol, isopropyl alcohol, and mixtures thereof, and most specific alcohol solvent is methanol. Exemplary ketone solvents include, but are not limited to, acetone, methyl ethyl ketone, methyl isobutyl ketone, methyl tert-butyl ketone and the like, and mixtures thereof. Exemplary nitrile solvents include, but are not limited to, acetonitrile, propionitrile and the like, and mixtures thereof. Exemplary ester solvents include, but are not limited to, ethyl acetate, isopropyl acetate, and the like and mixtures thereof. Exemplary chlorinated hydrocarbon solvents include, but are not limited to, methylene chloride, ethyl dichloride, chloroform, carbon tetrachloride, and mixtures thereof. Specific chlorinated hydrocarbon solvent is methylene chloride. Exemplary cyclic ether solvents include, but are not limited to, tetrahydrofuran, dioxane, and the like, and mixtures thereof. Exemplary aliphatic ether solvents include, but are not limited to, diethyl ether, diisopropyl ether, monoglyme, diglyme and the like, and mixtures thereof. Exemplary hydrocarbon solvents include, but are not limited to, n-pentane, n-hexane, n-heptane and isomers thereof, cyclohexane, toluene and xylene and the like, and mixtures thereof. Exemplary polar aprotic solvents include, but are not limited to, N,N-dimethylformamide, N,N-dimethylacetamide, dimethylsulfoxide, and mixtures thereof.

Step-(a) of providing a solution of fesoterodine fumarate includes dissolving fesoterodine fumarate in the solvent, or obtaining an existing solution from a previous processing step.

Preferably the fesoterodine fumarate is dissolved in the solvent at a temperature of below about boiling temperature of the solvent used, more preferably at about 20° C. to about 110° C., and still more preferably at about 25° C. to about 80° C.

The solution in step-(a) may also be prepared by reacting (+)-N,N-diisopropyl-3-(2-hydroxy-5-hydroxymethylphenyl)-3-phenylpropylamine with isobutyryl chloride in a suitable solvent, optionally in the presence of a base, under suitable conditions to produce a reaction mass containing crude fesoterodine free base followed by treatment with fumaric acid to produce a solution containing fesoterodine fumarate, or optionally subjecting the solution to usual work up such as washings, extractions etc., and dissolving the resulting fesoterodine fumarate in a suitable solvent at a temperature of below about boiling temperature of the solvent used, more preferably at about 25° C. to about 110° C. and still more preferably at about 25° C. to about 80° C.

The solution obtained in step-(a) may optionally be subjected to carbon treatment. The carbon treatment can be carried out by methods known in the art, for example by stirring the solution with finely powdered carbon at a temperature of below about 70° C. for at least 15 minutes, preferably at a temperature of about 40° C. to about 70° C. for at least 30 minutes; and filtering the resulting mixture through hyflo to obtain a filtrate containing fesoterodine fumarate by removing charcoal. Preferably, finely powdered carbon is an active carbon.

The solution obtained in step-(a) or step-(b) is optionally stirred at a temperature of about 30° C. to the reflux temperature of the solvent used for at least 20 minutes, and preferably at a temperature of about 40° C. to the reflux temperature of the solvent used from about 30 minutes to about 4 hours.

Removal of solvent in step-(c) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilisation or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying.

The distillation process can be performed at atmospheric pressure or reduced pressure. Preferably the solvent is removed at a pressure of about 760 mm Hg or less, more preferably at about 400 mm Hg or less, still more preferably at about 80 mm Hg or less, and most preferably from about 30 to about 80 mm Hg.

The substantially pure amorphous fesoterodine fumarate obtained by the above process may be further dried in, for example, Vacuum Tray Dryer, Rotocon Vacuum Dryer, Vacuum Paddle Dryer or pilot plant Rota vapor, to further lower residual solvents.

The total purity of the amorphous fesoterodine fumarate obtained by the process disclosed herein is of greater than about 99%, specifically greater than about 99.5%, and more specifically greater than about 99.9% as measured by HPLC.

According to another aspect of the present invention, there is provided a process for preparing stable amorphous form of fesoterodine fumarate comprising heating fesoterodine fumarate in a known crystalline form or in a mixture of known crystalline forms until the known form/s are converted to amorphous form.

The conversion to amorphous form occurs at a temperature of above about 95° C., preferably at about 98° C. to about 130° C., and more preferably at about 110° C. to about 120° C.

The heating is carried out for at least about 30 minutes, usually about 1 hour to about 15 hours and typically about 2 hours to about 12 hours.

After completion of the heating process, the resulting material is preferably cooled at a temperature of below about 50° C. for at least 30 minutes, and more preferably at a temperature of about 20° C. to about 40° C. from about 1 hour to 5 hours.

No racemization occurs during the heating of fesoterodine fumarate as evidenced by enantiomeric purity, which is same before and after heating.

In one embodiment, the substantially pure amorphous fesoterodine fumarate disclosed herein for use in the pharmaceutical compositions of the present invention, wherein 90 volume-percent of the particles (D₉₀) have a size of less than or equal to about 500 microns, specifically less than or equal to about 300 microns, more specifically less than or equal to about 200 microns, still more specifically less than or equal to about 100 microns, and most specifically less than or equal to about 15 microns.

In another embodiment, the particle sizes of substantially pure amorphous fesoterodine fumarate can be achieved by a mechanical process of reducing the size of particles which includes any one or more of cutting, chipping, crushing, milling, grinding, micronizing, trituration or other particle size reduction methods known in the art, to bring the solid state forms the desired particle size range.

According to another aspect of the present invention, there is provided a method for treating a patient suffering from urinary incontinence, comprising administering a therapeutically effective amount of the amorphous fesoterodine fumarate, or a pharmaceutical composition that comprises therapeutically effective amount of amorphous fesoterodine fumarate, along with pharmaceutically acceptable excipients.

According to another aspect of the present invention, there is provided pharmaceutical compositions comprising amorphous fesoterodine fumarate prepared according to processes of the present invention in any of its embodiments and one or more pharmaceutically acceptable excipients.

According to another aspect of the present invention, there is provided a process for preparing a pharmaceutical formulation comprising combining amorphous fesoterodine fumarate prepared according to processes of the present invention in any of its embodiments, with one or more pharmaceutically acceptable excipients.

Yet another embodiment of the present invention is directed to pharmaceutical compositions comprising at least a therapeutically effective amount of substantially pure amorphous fesoterodine fumarate of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The pure amorphous fesoterodine fumarate of the present invention may also be administered as suppositories, ophthalmic ointments and suspensions, and parenteral suspensions, which are administered by other routes. The dosage forms may contain amorphous fesoterodine fumarate of the present invention as is or, alternatively, may contain amorphous fesoterodine fumarate of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain amorphous fesoterodine fumarate of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The following examples are provided to enable one skilled in the art to practice the invention and are merely illustrate the process of this invention. However, it is not intended in any way to limit the scope of the present invention.

### Experimental:

### Chromatographic Parameters:

Column: Kromasil C18 150×4.6 mm×3.5 μm (Part No:-E-32561)

### Make: AKZONOBEL.

### Detector: UV at 220 nm

Flow rate: 0.8 mL/min
Injection volume: 10.0 μL
Run time: 60 min
Column temperature: 40° C.
Sample temperature: Ambient
Diluent: Transfer 2mL of acetonitrile in to 50 mL volumetric flask then make up to volume with Mobile phase-A

The products of the following Examples 1-3 each gave an powder X-ray diffraction pattern and infra-red spectrum as shown in FIGS. 1 and 2. The products had a shelf life suitable for commercial use and were suitable for rapid absorption after oral administration.

### EXAMPLES

### Example 1

### Process for Preparing Amorphous Fesoterodine Fumarate

Fesoterodine fumarate (2.0 g) was heated at 110° C. to 115° C. under nitrogen atmosphere to melt. The molten fesoterodine fumarate was allowed to cool at 25-30° C. for 1 hour to give amorphous fesoterodine fumarate quantitatively.

### Example 2

### Process for Preparing Amorphous Fesoterodine Fumarate

Fesoterodine fumarate (2.0 g) was dissolved in a mixture of dichloromethane (35 ml) and methanol (15 ml) at 25-30° C. to obtain clear solution. The solvents were removed completely under vacuum at 40° C. and then dried for 12 hours to give 1.8 g of fesoterodine fumarate in amorphous form (HPLC Purity: 99.8%).

### Example 3

### Process for Preparing Amorphous Fesoterodine Fumarate

Fesoterodine fumarate (2.0 g) was dissolved in a mixture of dichloromethane (35 ml) and methanol (15 ml) at 25-30° C. to obtain clear solution. The solution was filtered through Hyflo bed. The resulting clear solution was concentrated to dryness using laboratory spray dryer (Jay Instruments & Systems Pvt. Ltd. India, Model-LSD-48 mini Spray Dryer) to give 1.5 g of amorphous fesoterodine fumarate (HPLC Purity: 99.6%).

1. Amorphous form of fesoterodine fumarate characterized by at least one or more of the following properties:
i) a powder XRD pattern substantially in accordance with FIG. 1;
ii) an IR spectrum substantially in accordance with FIG. 2; and
iii) an IR spectrum having absorption bands at about 3399, 3028, 2977, 2940, 2877, 1751, 1702, 1680, 1616, 1495, 1468, 1387, 1226, 1179, 1129, 1095, 983, 867, 804 and 701±1 cm⁻¹ substantially as depicted in FIG. 2.

2. A process for the preparation of amorphous fesoterodine fumarate of claim 1, comprising:
a) providing a solution of fesoterodine fumarate in a solvent or a mixture of solvents capable of dissolving fesoterodine fumarate, wherein the solvent or the solvent mixture is selected from the group consisting of water, alcohols, ketones, chlorinated hydrocarbons, hydrocarbons, nitriles, esters, cyclic ethers, aliphatic ethers, polar aprotic solvents, and mixtures thereof;
b) optionally, filtering the solvent solution to remove any extraneous matter; and
c) substantially removing the solvent from the solution to afford amorphous form of fesoterodine fumarate, wherein the removal of the solvent is accomplished by complete evaporation of the solvent, spray drying, vacuum drying, lyophilization or freeze drying, or a combination thereof.

3. (canceled)

4. The process of claim 2, wherein the solvent or the solvent mixture used in step-(a) is selected from the group consisting of water, methanol, ethanol, propanol, isopropanol, butanol, amyl alcohol, hexanol, acetone, methyl ethyl ketone, methyl isobutyl ketone, methyl tert-butyl ketone, acetonitrile, propionitrile, ethyl acetate, isopropyl acetate, methylene chloride, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof.

5. The process of claim 2, wherein the solvent mixture used in step-(a) is a mixture of methylene chloride and an alcohol solvent.

6. The process of claim 5, wherein the solvent mixture is a mixture of methylene chloride and methanol.

7. The process of claim 2, wherein the solution in step-(a) is provided by dissolving fesoterodine fumarate in the solvent at a temperature of below about boiling temperature of the solvent used; wherein the solution obtained in step-(a) is optionally subjected to carbon treatment; wherein the solution obtained in step-(a) or step-(b) is optionally stirred at a temperature of about 30° C. to the reflux temperature of the solvent used for at least 20 minutes; and wherein the amorphous fesoterodine fumarate obtained has a total purity of greater than about 99% as measured by HPLC.

8. The process of claim 7, wherein the fesoterodine fumarate is dissolved in the solvent at a temperature of about 20° C. to about 110° C.; wherein the solution obtained in step-(a) or step-(b) is stirred at a temperature of about 40° C. to the reflux temperature of the solvent used from about 30 minutes to about 4 hours; and wherein the amorphous fesoterodine fumarate has a total purity of greater than about 99.9% as measured by HPLC.

9-16. (canceled)

17. A process for preparing amorphous fesoterodine fumarate of claim 1, comprising heating fesoterodine fumarate in a known crystalline form or in a mixture of known crystalline forms until the known form/s are converted to amorphous form.

18. The process of claim 17, wherein the fesoterodine fumarate is heated at a temperature of above about 95° C. for at least 30 minutes; and wherein the material obtained after heating process is cooled at a temperature of below about 50° C. for at least 30 minutes.

19. The process of claim 18, wherein the fesoterodine fumarate is heated at a temperature of about 98° C. to about 130° C. from about 1 hour to about 15 hours; and wherein the material obtained after heating process is cooled at a temperature of about 20° C. to about 40° C. from about 1 hour to 5 hours.

20-22. (canceled)

23. A pharmaceutical composition comprising amorphous fesoterodine fumarate of claim 1 and one or more pharmaceutically acceptable excipients.

24. (canceled)

25. (canceled)

26. The pharmaceutical composition of claim 23, wherein the pharmaceutical composition is selected from dosage forms comprising liquid, powder, elixir and injectable solution.

27. The pharmaceutical composition of claim 26, wherein the pharmaceutical composition is selected from a solid dosage form and an oral suspension.

28. The pharmaceutical composition of claim 23, comprising particles of pure amorphous fesoterodine fumarate, wherein 90 volume-% of the particles (D₉₀) have a size of less than or equal to about 400 microns.

29. The pharmaceutical composition of claim 28, wherein the 90 volume-% of the particles (D₉₀) have a size of less than or equal to about 300 microns, less than or equal to about 200 microns, less than or equal to about 100 microns, less than or equal to about 60 microns, or less than or equal to about 15 microns.

30-33. (canceled)

34. A method of treating a patient suffering from urinary incontinence, comprising administering a therapeutically effective amount of the amorphous fesoterodine fumarate of claim 1, or a pharmaceutical composition that comprises a therapeutically effective amount of amorphous fesoterodine fumarate, along with pharmaceutically acceptable excipients.
